(19) United States
(12) Patent Application Publication
(10) Pub. No.: US 2002/0198161 A1
(43) Pub. Date: Dec. 26, 2002

(54) THERAPEUTIC USES OF ANTIOXIDANTS

(76) Inventors: DOUGLAS E. BRASH, KILLINGWORTH, CT (US); MING LIU, HAMDEN, CT (US)

Correspondence Address: PENNIE & EDMONDS, 1155 AVENUE OF THE AMERICAS, NEW YORK, NY, 100362711

(21) Appl. No.: 09/027,204

(22) Filed: Feb. 20, 1998

Related U.S. Application Data
(60) Provisional application No. 60/038,707, filed on Feb. 20, 1997.

(51) Int. Cl. A61K 48/00; A61K 31/545; C12N 7/00

(52) U.S. Cl. 514/44; 514/208; 424/277.1; 435/235.1; 435/325

(57) ABSTRACT

The present invention relates to methods and compositions useful for cancer and precancer therapy utilizing sulphur-containing antioxidants. In particular, the present invention relates to methods and compositions which selectively induce apoptosis in cells of cancers or precancers.

The present application claims benefit under 35 U.S.C. §119(e) to U.S. provisional application Ser. No. 60/038,707, filed Feb. 20, 1997, the contents of which are incorporated herein by reference in their entirety.

This invention was made with government support under grant number CA55737 awarded by the National Institutes of Health. The government has certain rights in the invention.

### 1. INTRODUCTION

The present invention relates to methods and compositions useful for cancer and precancer therapy utilizing sulphur-containing antioxidants. In particular, the present invention relates to methods and compositions which selectively induce apoptosis in cells of cancers or precancers.

### 2. BACKGROUND OF THE INVENTION

Antioxidants have a wide range of biochemical activities. These include inhibiting the generation of reactive oxygen species, directly or indirectly scavenging free radicals, and altering the intracellular redox potential (Miquel, J. et al., 1989, CRC Handbook of Free Radicals and Antioxidants in Biomedicine, Boca Raton: CRC Press). For example, some antioxidants have been used as inhibitors of apoptosis, because apoptosis was at first thought to be mediated by oxidative stress (Hockenbery, D. M. et al., 1993, Cell 75:241-251; Verhaegen, S. et al., 1995, Biochem. Pharmacol. 7:1021-1029; and Lotem, J. et al., 1996, Proc. Natl. Acad. Sci. USA 93:9166-9171). Reactive oxygen species are not, however, always required to induce apoptosis (Shimizu, S. et al., 1995, Nature 374:811-813; Jacobson, M. D. & Raff, M. C., 1995, Nature 374:814-816).

In contrast, antioxidants have been shown to trigger apoptosis in smooth muscle cells (Tsai, J. et al, 1996, J. Biol. Chem. 271:3667-3670). Furthermore, antioxidants have been reported to exhibit a “biphasic” (that is, inductive or inhibitory) influence over apoptosis, depending on the antioxidant concentration utilized (Held, K. D. et al., 1996, Radiation Res. 145:542-553).

Antioxidants have also been utilized as radioprotectants to protect cells from radiation induced DNA damage, chromosomal aberrations, cytotoxicity and mutagenesis (Grdina, D. J. et al., 1985, Carcinogenesis 6:929-931; Smolk, G. D. et al., 1988, Cancer Research 48:3641-3647; Grdina, D. J. et al., 1989, Radiation Res. 117:500-510; and Grdina, D. J. et al., 1992, Carcinogenesis 13:811-814).

Pro-oxidant states have been considered to be contributing factors for tumorigenesis (Cerutti, P. A., 1985, Science 227:375-381). Correspondingly, antioxidants have been proposed as cancer preventative agents (Steele, V. E. et al., 1990, Cancer Res. 50:2068-2074; O'Brien, P., 1994, in D. Armstrong (ed.) Free Radicals in Diagnostic Medicine, pp. 215-239, New York: Plenum Press).

For example, the antioxidant N-acetylcysteine (NAC) has been reported to exhibit antimutagenic, anticarcinogenic and chemopreventive activities (Steele, V. E. et al., 1990, Cancer Res. 50:2068-2074; Flora, S. D. et al., 1986, Cancer Letters 32:235-241; Rostein, J. B. & Slaga, T. J., 1988, Mutation Research 202:421-427; Pereira, M. A. & Khoury, M. D., 1991, Cancer Letter 61:27-33; Flora, S. D. et al., 1992, in Wattenberg, L. et al., (eds.), Cancer Chemoprevention, pp. 183-194, Boca Raton, Fla.: CRC Press; and Izzotti, A. et al., 1994, Cancer Res. 54:1994s-1998s).

The tumor suppressor protein p53 is also known to play an important role in inhibiting tumorigenesis. This transcription factor is involved in cell cycle arrest and apoptosis after DNA damage (Ko, L. J. & Prives, C., 1996, Genes & Dev. 10:1054-1072; Levine, A. J., 1997, Cell 88:323-331). Manipulating p53-mediated pathways has thus been a major focus for cancer therapy (Ko, L. J. & Prives, C., 1996, Genes & Dev. 10:1054-1072; Levine, A. J., 1997, Cell 88:323-331). For example, restoring expression of wild-type p53 renders cells more sensitive to spontaneous or chemotherapy-induced apoptosis (Fujiwara, T. et al., 1994, Cancer Res. 54:2287-2291; Liu, T. J. et al., 1995, Cancer Res. 55:3117-3122). There is also a good correlation between a tumor's p53 functional status and its response to some chemotherapeutic agents (Lowe, S. W. et al., 1993, Cell 74:957-967; Lowe, S. W. et al., 1994, Science 266:807-810; and O'Connor, P. M. et al., 1997, Cancer Res. 57:4285-4300).

In the past few years, p53 function has been reported to be redox-regulatable in vitro through its cysteine residues (Hainaut, P. & Milner, J., 1993, Cancer Res. 53:4469-4473; Hupp, T. R. et al., 1993, Nucleic Acid Research 21:3167-3174; and Rainwater, R. et al., 1995, Mol. Cell. Biol. 15:3892-3903).

Unlike the antioxidant uses described above, the present invention provides methods and compositions for the treatment and removal of cells of already preexisting cancers and precancers.

Citation of references hereinabove shall not be construed as an admission that such references are prior art to the present invention.

### 3. SUMMARY OF THE INVENTION

The present invention relates, first, to methods and compositions useful for cancer and precancer therapeutic treatment utilizing sulphur-containing antioxidants (S-antioxidants). First, the present invention relates to methods and compositions which selectively induce apoptosis in cells of cancers or precancers.

In one embodiment, the methods of the present invention comprise selectively inducing apoptosis of precancer cells by administering an effective amount of an S-antioxidant to a subject. In a preferred embodiment, the S-antioxidant is topically administered.

In another embodiment, the methods of the present invention comprise selectively inducing apoptosis in cancer cells by administering an effective amount of an S-antioxidant to a subject. In a preferred embodiment, the S-antioxidant is topically administered.

In yet another embodiment, the methods of the present invention comprise reducing the number of cancer cells present in a subject by administering an S-antioxidant to the subject as an adjunct to chemotherapy or radiation therapies such that the susceptibility of the cancer cells to apoptosis is enhanced relative to the non-cancer cells of the subject.

In still another embodiment, the methods of the present invention comprise administering the S-antioxidants of the invention as an adjunct to p53 therapy, including p53 gene therapy.

In another embodiment of the invention, the methods of the present invention comprise administering the S-antioxidants of the invention to selectively induce cells which arise in hyperproliferative or benign dysproliferative disorders.

The present invention also relates to methods for selective cell cycle arrest comprising contacting the cell with a sulphur-containing antioxidant.

It is also contemplated that the methods of the invention can be utilized to reduce or inhibit tumor vascularization, or to induce differentiation in cancer cells. It is further contemplated that the S-antioxidants of the invention can be administered to inhibit HIV-1 replication.

The cancer or precancer cells in which apoptosis is induced are generally ones which exhibit at least one functional p53 allele. It is to be noted that in certain instances, administration of the S-antioxidant results in restoration of mutant p53 protein conformation and/or activity to a functional state. Further, it is noted that an endogenous functional p53 allele is not necessary for methods comprising p53 therapy, including p53 gene therapy.

The S-antioxidants of the present invention are ones which exhibit an ability to selectively induce apoptosis in cancer or precancer cells relative to non-cancerous or non-precancerous cells. Such S-antioxidants can, for example, be thiol-containing antioxidants. Alternatively, such S-antioxidants can, for example, be sulphur-containing antioxidants which exhibit one or more sulphur moieties within a ring structure. Preferred S-antioxidants include, for example, N-acetylcysteine (NAC) and 2,3-dimercaptopropanol (DMP), L-2-oxo-4-thiazolidinecarboxylate (OTC) and lipoic acid.

This invention is based, in part, on the discovery that administration of S-antioxidants leads to selective glutathione (GSH)-independent apoptosis in transformed cells. Apoptosis is shown to be selective in that corresponding normal, non-transformed cells do not undergo S-antioxidant-induced cell death. These results are described in the Example presented in Section 6, below.

The invention is further based on the discovery that administration of S-antioxidants leads to prolonged transition through G₁ phase. This cell cycle arrest appears to be influenced by an increase in p²¹ expression. These results are described in the Example presented in Section 7, below.

While not wishing to be bound by any particular theory, it appears that the apoptosis is mediated by an increase in p53 protein levels. The increase is shown to be due to an increase in the rate of protein synthesis, not transcription or protein stabilization. The increase in p53 does not appear to be sufficient for apoptosis, however, in that the increase is seen in both normal and transformed cells.

The present invention may be understood more fully by reference to the detailed description and illustrative examples which are intended to exemplify non-limiting embodiments of the invention.

### 3.1. DEFINITIONS

As used herein, “antioxidant” means a compound which can prevent oxidation of a substrate. The antioxidants utilized herein are sulphur-containing antioxidants, which can be referred to herein as “S-antioxidants.”

As used herein, “precancer” means a condition known or suspected to precede progression, or exhibit the potential to progress, to neoplasia or cancer, in particular, where non-neoplastic cell growth consisting of hyperplasia, metaplasia, or dysplasia has occurred.

As used herein, “apoptosis” means a form of cell death characterized by cell shrinkage, detachment, and nuclear and cellular fragmentation.

As used herein, “selectively induce apoptosis” means induction of a higher level of apoptosis in one group of cells (i.e., cancer or precancer cells) relative to a second group of cells (i.e., the corresponding non-cancer or precancer cells).

As used herein, “pharmaceutical” means a formulation to be administered, for example administered to the skin, which renders a benefit or an effect of treating or preventing an abnormal biological condition or a disease.

As used herein, “safe and effective amount” means an amount of a compound or composition, sufficient to significantly induce a positive modification (e.g., induction of apoptosis) in the condition to be treated, but low enough to avoid serious side effects (at a reasonable benefit/risk ratio). The safe and effective amount of the compound or composition will vary with the particular condition being treated, the age and physical condition of the patient being treated, the severity of the condition, the nature of concurrent treatment, the specific compound, compounds or composition employed, the particular pharmaceutically-acceptable carrier utilized, and like factors within the knowledge and expertise of the attending physician or health care provider.

### 4. BRIEF DESCRIPTION OF THE FIGURES

FIGS. 1A-1B. Induction of apoptosis by NAC in 308 papilloma cells. FIG. 1A: cell viability measured by trypan blue exclusion; results from three independent experiments (mean +/−SD). FIG. 1B: apoptosis-associated DNA strand breaks in cells exposed to 20 mM NAC for 24 h, visualized by fluorescent in situ end-labeling. Both photographs have the same magnification. In NAC-treated cells, apoptotic nuclei are indicated by arrows and other cells have a condensed morphology. Cytoplasmic background results from RNA staining.

FIGS. 2A-2C. p53-dependent apoptosis by NAC is selective for transformed cells. FIG. 2A: viability of normal (MEF) and transformed (tMEF) cells measured by trypan blue exclusion after treatment with NAC for 24 h. FIG. 2B: analysis of DNA fragmentation from tMEF cells exposed to NAC. FIG. 2C: immunohistochemical staining of p53 protein in p53+/+ MEF cells after 5 h exposure to 20 mM NAC.

FIG. 3. NAC induces p53 protein through an increased p53 translation rate in 308 cells. FIG. 3A: time course of p53 protein induction in total cell lysates by 20 mM NAC (upper); dose-dependent p53 protein induction by NAC after 5 h treatment (middle), Northern blot of total RNA from cells untreated or exposed to 20 mM NAC for 5 h (lower). FIG. 3B: synthesis of p53 protein in cells at 5 h post-treatment with 20 mM NAC. Arbitrary units of p53 protein band density on the autoradiogram were plotted. FIG. 3C: p53 protein half-life in cells at 5 h post-treatment with 20 mM NAC. For FIGS. 3B and 3C, data represent one of two similar experiments.

FIG. 4. p53-dependent apoptosis induced by 50 μM 2,3-dimercaptopropanol (DMP) and 20 mM L-2-oxo-4-thiazolidinecarboxylate (OTC) in transformed MEF. FIG. 4A: cell viability. FIG. 4B: DNA fragmentation analysis at 24 h posttreatment. FIG. 4C: flow-cytometry analysis at 48 h posttreatment. Box R1 represents viable cells. Box R2 shows apoptotic cells, defined as having sub-G1 DNA fluorescence (y-axis) and a forward angle light scatter (x-axis)≦cells in G1 phase. All data represent 2-3 independent experiments.

FIG. 5. Sulfur-free antioxidants tocopherol acetate (200 μM), Trolox (200 μM), and BHA (100 μM) do not induce cell death in tMEF p53+/+ cells. Antioxidants were first dissolved in ethanol, and then added to the medium. The final concentration of ethanol in the medium is 1:2,000 (v/v). At 48 h, cell viability was measured by trypan blue exclusion. All data represent 2 independent experiments.

FIG. 6. Analysis of GSH, total thiols, and viability in tMEF p53+/+ cells treated with BSO and/or NAC. Cells treated with both compounds were preincubated with medium containing 20 μM BSO for 1 h and then treated with medium containing 20 μM BSO and 20 mM NAC for 5 h (GSH and thiols) or for 24 h (viability).

FIG. 7. Twin antioxidant pathways for apoptosis. The present data indicate that sulfur-containing antioxidants alter intracellular thiol levels, elevate p53 protein, and induce apoptosis in transformed cells (left). Since p53 rises even in normal cells (FIG. 2C), apoptosis requires an additional transformation-related signal (right). For example, cells with aberrant cell cycles caused by viral or transgenic inactivation of Rb undergo apoptosis; this apoptosis requires p53 (White, E., 1994, Nature 371:21-22). Evidently, apoptosis requires both a cell cycle abnormality signal and a detector. S-antioxidants augment the p53-dependent detector via redox regulation. Such a signal could synergize with cell cycle abnormalities already existing from transformation or chemotherapy.

### 5. DETAILED DESCRIPTION OF THE INVENTION

### 5.1. S-antioxidant Compounds and Compositions

The antioxidants of the present invention are ones which exhibit an ability to selectively induce apoptosis in cancer or precancer cells relative to non-cancerous or non-precancerous cells. In general, the antioxidants of the present invention are sulphur-containing antioxidants (“S-antioxidants”). The S-antioxidants of the present invention are ones which exhibit an ability to selectively induce apoptosis in cancer or precancer cells relative to non-cancerous or non-precancerous cells.

Such S-antioxidants can, for example, be thiol-containing antioxidants. Alternatively, such S-antioxidants can, for example, be sulphur-containing antioxidants which exhibit one or more sulphur moieties within a ring structure. For example, such antioxidants can include, but are not limited to, dithioethiones, diallyl sulphide, and the like. Preferred S-antioxidants include, for example, N-acetylcysteine (NAC), 2,3-dimercaptopropanol (DMP), L-2-oxo-4-thiazolidinecarboxylate (OTC) and lipoic acid.

Described below, are in vitro, ex vivo and in vivo assays (Section 5.1.1) which can be utilized to routinely identify S-antioxidant compounds which can be used as part of the methods of the present invention, and pharmaceutical compositions and routes of administration of such S-antioxidant compounds (Section 5.1.2).

### 5.1.1. Assays for Identifying S-antioxidant Compounds

In vitro and in vivo assays described herein can be used to routinely identify S-antioxidants which can be utilized as part of the methods of the present invention.

First, in vitro assays can be utilized for testing the usefulness of a candidate S-antioxidant. Such in vitro assays can include, for example, testing the ability of a candidate S-antioxidant to induce apoptosis in paired sets of normal and transformed cells. Such paired sets of cells differ in whichever feature is utilized to transform the transformed cells.

In one example, the paired cells are fibroblasts, such as embryonic fibroblasts obtained from inbred animals (e.g., mice), which differ only in that the transformed cells contain E1a and ras oncogene constructs. The cells should exhibit at least one functional p53 allele.

In certain instances, for example when it is desired to determine whether an S-antioxidant exhibits an ability to restore p53 activity, cells which are homozygous for mutant p53 alleles can be utilized. In addition, cells of the type described above but lacking a functional p53 allele can be utilized along with the paired sets of cells to determine whether the results generated by the candidate S-antioxidant are p53 dependent.

The cells are contacted with the S-antioxidant at a range of concentrations for a time sufficient to induce apoptosis, and are assayed for the signs of apoptosis. Tests for apoptosis are well known to those of skill in the art and include, for example, analysis of DNA strand breaks (see, e.g., Ziegler, A. et al., 1994, Nature 372:773-776; and Lowe, S. W. et al., 1993, Cell 74:957-967), and morphological analysis of, for example, cell shrinkage, nuclear condensation and nuclear and cellular fragmentation.

Those S-antioxidants which selectively induce apoptosis in transformed cells represent compounds which can be utilized as part of the methods of the present invention. As used herein, “selectively induce apoptosis” means induction of a higher level of apoptosis in one group of cells (i.e., cancer or precancer cells) relative to a second group of cells (i.e., the corresponding non-cancer or precancer cells).

Alternatively, in instances in which paired sets of normal and transformed cells do not exist, in vitro assays can compare normal primary cell lines against closely matched (that is, closely matched genotypically and/or phenotypically) tumor cell lines. As above, the cells are contacted with a candidate S-antioxidant at a range of concentrations for a time sufficient to induce apoptosis, and are assayed for the signs of apoptosis.

For example, human primary cell lines can be compared to human tumor cells lines, e.g., cell lines of the NCI cell panel (O'Connor, P. M. et al., 1997, Cancer Res. 57:4285-4300). In general, such cell lines should exhibit at least one functional p53 allele. In particular, cell lines to be tested can include, for example, SK-MEL-5 (melanoma), MCF-5 (breast), A549 (lung) and HCT-116 (colon) cell lines. Such cells can be compared to, for example, primary closely matched primary cell lines. In the case of skin cancer-related cells, for example, appropriate transformed cell lines can be compared to appropriate melanocytes, keratinocytes and fibroblasts derived from human foreskins.

Such tests can also be performed using cell lines which by virtue of, for example, deletions, frameshift mutations or splicing mutations, lack p53 function (“p53”). Among the human cell lines which can be assayed are, for example, MCF-7/ADR-RES (breast), EKVX, NCI-H522, HOP-62, CaLu-1 (lung), and HCC-2998 (colon). Results obtained in such cells can be compared to results obtained in cells exhibiting p53 function to determine whether the effects of the candidate S-antioxidant are p53-dependent. Such results can also be used to determine whether the candidate S-antioxidant restores p53 function to mutant p53 alleles.

Alternatively, p53 dependent S-antioxidant activity can be assayed using p53⁻ transformed cells transiently transfected with vectors expressing normal p53. In such a system, cells transfected with p53 or with vector alone constitute a matched pair of directly comparable cells. The p53⁻ transformed recipient cells are chosen as above. The p53⁻ transformed cells are less susceptible to apoptosis than the tumor cell lines exhibiting p53 activity. Transfecting normal p53 into a p53⁻ cell restores sensitivity.

For example, for purposes of assaying squamous cell carcinomas, p53⁻ transformed cell lines comprise SCC-13 and HaCaT cell lines.

Ex vivo assays for tumorigenicity can also be utilized to identify candidate S-antioxidants. For example, standard ex vivo soft agar models of tumorigenicity can be utilized.

Cells in the soft agar models are contacted to a candidate S-antioxidant at a range of concentrations for a time sufficient to induce apoptosis. Cells are then assayed for signs of apoptosis and reduced colony formation.

In vivo assays can also be utilized to used to routinely identify S-antioxidant compounds which can be utilized as part of the methods of the present invention without undue experimentation.

For example, test subjects can be treated in such a manner as to induce precancer or cancer lesions, e.g., clones of mutant cells. Such treatments can include, but are not limited to UV irradiation, preferably UVB irradiation. For example, UVB irradiation (e.g., daily irradiation of shaved skin for a 2-4 week period) can induce mutant clones on mouse skin. Alternatively, UVB irradiation (e g., daily irradiation of shaved skin for a 6 week period) can induce actinic keratoses on mouse skin.

Another example would be in vivo athymic nude (nu/nu) mice models containing appropriate human tumor cell xenografts (see, e.g., Chinery et al., 1997, Nature Medicine 3:1233-1241).

A candidate S-antioxidant at a range of concentrations is administered (e.g., topically or by injection) to a sample of treated animals in a manner which places the S-antioxidant in contact with the lesions (e.g., the clones of mutant cells) for a time sufficient to induce apoptosis. Animals are then assayed for the signs of mutant cell apoptosis and lesion regression.

### 5.1.2. S-antioxidant Pharmaceutical Compositions and Routes of Administration

The pharmaceutical compositions of the present invention are those which, when administered, for example when administered to the skin, to a subject in a safe and effective amount render a benefit or an effect of treating a condition, e.g., a precancer or cancer condition. In particular, such benefit can comprise selective induction of apoptosis of precancerous or cancerous cells. Benefits or effects of treatment may be either in the short term or the long term. Section 5.2, below, describes specific uses for the S-antioxidant compounds and compositions of the invention, and methods for routinely determining S-antioxidant dosages. The subject is preferably an animal, including but not limited to animals such as cows, pigs, horses, chickens, cats, dogs, etc., and is preferably a mammal, and most preferably human. In a specific embodiment, a non-human mammal is the subject.

As used herein, the term “safe and effective amount” means an amount of compound or composition sufficient to significantly induce a positive modification (e.g., induction of apoptosis) of the condition to be treated, but low enough to avoid serious side effects (at a reasonable benefit/risk ratio), within the scope of sound medical judgment. The safe and effective amount of the compound or composition will vary with the particular condition being treated, the age and physical condition of the patient being treated, the severity of the condition, the duration of the treatment, the nature of concurrent therapy, the specific compound, compounds or compositions employed, the particular pharmaceutically acceptable carrier utilized, and like factors within the knowledge and expertise of the attending physician or health care provider.

The S-antioxidant compounds of the present invention can be synthesized in accordance with standard chemical techniques using readily/commercially available starting materials. Alternatively, the S-antioxidants of the present invention can be prepared from semisynthetic methods. Still further, the S-antioxidants can be purified or partially purified from natural sources. In a preferred aspect, the S-antioxidant is substantially purified.

In a specific embodiment, the S-antioxidant pharmaceutical compositions further comprise a functional p53 polypeptide. For example, such a p53 polypeptide can comprise a full length wild type, e.g., human p53 polypeptide. Such a p53 polypeptide can, for example, also comprise a portion of a p53 polypeptide, such as a human p53 polypeptide, which retains p53 function.

In another specific embodiment, the S-antioxidant pharmaceutical compositions further comprise a nucleic acid encoding a functional p53 polypeptide. For example, such a nucleic acid can encode p53 polypeptide comprising a full length wild type, e.g., human p53 polypeptide. Such a nucleic acid can, for example, also encode a molecule comprising a portion of a p53 polypeptide, such as a human p53 polypeptide, which retains p53 function.

Both p53 polypeptides and nucleic acid molecules encoding such polypeptides are well known to those of skill in the art. See, for example, WO 97/10007; U.S. Pat. No. 5,573,925; and WO 95/11301, which are hereby incorporated by reference in their entirety.

Pharmaceutical compositions for use in accordance with the present invention may be formulated in conventional manner using one or more physiologically acceptable carriers or excipients. Such pharmaceutical compositions will contain a safe and effective amount of the S-antioxidant, preferably in purified form, together with a suitable amount of carrier so as to provide the form for proper administration to the patient. The formulation should suit the mode of administration.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the S-antioxidant is administered.

Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

These compositions can take the form of solutions, suspensions, emulsion, tablets, pills, capsules, powders, suppositories, sustained-release formulations, lotions, tincures, creams, emulsions, mousses, sprays, foams, powders, gels, ointments and the like.

The S-antioxidants of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In particular, the S-antioxidant compounds and their physiologically acceptable salts and solvates may be formulated for administration by inhalation or insufflation (either through the mouth or the nose) or oral, buccal, rectal, transmucosal, intralesional, intestinal or topical administration; parenteral delivery, including intramuscular, subuctaneous, intramedullary injections, as well as intrathecal, direct intraventricular, intravenous, intraperitoneal, intranasal, or intraocular injections. Administration can be systemic. Alternatively, one may administer the S-antioxidant compound locally.

In addition, it may be desirable to introduce the pharmaceutical S-antioxidant compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical S-antioxidant compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection or application at or on the site (or former site) of a malignant tumor or neoplastic or pre-neoplastic tissue directly.

In another embodiment, the S-antioxidant can be delivered in a vesicle, in particular a liposome (see, e.g., Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the S-antioxidants can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active S-antioxidant compound.

For buccal administration the compositions may take the form of tablets or lozenges formulated in conventional manner.

For administration by inhalation, the S-antioxidant compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

In a specific embodiment, the S-antioxidant composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The S-antioxidant compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The S-antioxidant compounds may also be formulated in rectal compositions such as suppositories or retention enemas, e.g., containing conventional suppository bases and binders such as cocoa butter or other glycerides.

In addition to the formulations described previously, the S-antioxidant compounds may also be formulated as a depot preparation. Such long acting formulations may be administered by implantation (for example subcutaneously or intramuscularly) or by intramuscular injection. Thus, for example, the S-antioxidant compounds may be formulated with suitable polymeric or hydrophobic materials (for example as an emulsion in an acceptable oil) or ion exchange resins, or as sparingly soluble derivatives, for example, as a sparingly soluble salt.

In instances in which skin cancers or precancers are being treated, the preferred mode of administration is topical. The pharmaceutical S-antioxidant compositions of the present invention intended for topical application may contain carrier, excipient or vehicle ingredients such as, for example, water, acetone, ethanol, ethylene glycol, propylene glycol, butane-1,3-diol, isopropyl myristate, isopropyl palmitate, mineral oil, and mixtures thereof to form lotions, tincures, creams, emulsions, mousses, sprays, foams, powders, gels or ointments which are non-toxic and pharmaceutically or dermatalogically acceptable. Additionally, moisturizers or humecants can be added to the present compositions if desired. Examples of such additional ingredients useful for such pharmaceutical compositions and actual methods for preparing pharmaceutical compositions can be found in Remington's Pharmaceutical Sciences, Eighteenth Edition, A. R. Gennaro, Ed., Mack Publishing Co. Easton Pa., 1990, which is incorporated herein by reference in its entirety.

The S-antioxidant compositions of the present invention can also be adapted for topical cosmetic application, for example, as part of a sunscreen formulation. The S-antioxidant compounds of the present invention can be formulated into suitable cosmetic compositions depending on the particular use for which it is intended.

The compositions of the present invention useful for topical application may contain additional ingredients such as carrier, excipient or vehicle ingredients such as, for example, water, acetone, ethanol, ethylene glycol, alphahydroxy acids, propylene glycol, butane-1,3-diol, isopropyl myristate, isopropyl palmitate, mineral oil, fragrances, preservatives, vitamins and mixtures thereof to form lotions, tinctures, creams, emulsions, gels or ointments, which are non-toxic and pharmaceutically or dermatologically acceptable. Additionally moisturizers, humectants, emollients, fragrances and pigments can be added to the present composition if desired. Examples of such additional ingredients can be found in Remington's Pharmaceutical Sciences, Eighteenth Edition, A. R. Gennaro, Ed., Mack Publishing Co. Easton Pa., 1990, or in the CTFA International Cosmetics Ingredients Dictionary (4th Edition).

Most compositions of the present invention may be formulated as solution, gel, lotion, cream, or ointment in a cosmetically acceptable form. Actual methods for preparing cosmetic compositions are known or apparent to those skilled in the art and are described in detail in for example, Remington's Pharmaceutical Sciences, 17th ed., Mack Publishing Company, Easton, Pa. (1990), which is incorporated herein by reference.

The invention also provides a pharmaceutical or cosmetic pack or kit comprising one or more containers filled with one or more of the ingredients of the pharmaceutical S-antioxidant compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.1.2.1. S-antioxidant Effective Doses

The amount of the S-antioxidant of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

However, toxicity and therapeutic efficacy of dosages of the S-antioxidant compounds identified via the assays described, above in Section 5.1.1, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

S-antioxidant compounds which exhibit large therapeutic indices are preferred. While S-antioxidant compounds that exhibit toxic side effects may be used, care should be taken to design a delivery system that targets such compounds to the site of affected tissue in order to minimize potential damage to uninfected cells and, thereby, reduce side effects.

The data obtained from in vitro, ex vivo and in vivo assays such as those described, above, in Section 5.1.1, can be used in formulating a range of dosage for use in humans. Effective doses may be extrapolated from dose-response curves derived from such assays. In general, such dosages should approximate whole body equivalent dosage level of the effective concentration identified via such tests.

For topical administration, effective dosages identified via in vitro or animal tests can be used to determine the dosage to be administered to a human subject such that the S-antioxidant concentration approximates the effective concentration identified via tests. The actual dosage may vary within this range depending upon the topical pharmaceutical composition chosen for such topical application.

For internal administration, the dosage of such S-antioxidant compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In general, the dosage range for the S-antioxidants of the invention will range from about 10 μM to about 50 mM.

### 5.2. Uses of the S-antioxidant Compounds and Compositions

The S-antioxidant compounds that are determined to selectively induce apoptosis, and pharmaceutical compositions thereof, can be used for a variety of purposes, as described herein.

The safe and effective amount of the S-antioxidant compound or composition will vary with the particular condition being treated, the age and physical condition of the patient being treated, the severity of the condition, the nature of concurrent treatment, the specific compound, compounds or composition employed, the particular pharmaceutically-acceptable carrier utilized, and like factors within the knowledge, and expertise of the attending physician or health care provider. The teaching provided in Section 5.2.1.2, above, however, can successfully be utilized as a guide to routinely determining useful S-antioxidant dosage ranges.

In particular, the methods of the present invention are useful, first, for selectively inducing apoptosis of precancer cells by administering a safe and effective amount of an S-antioxidant to a subject. Administration results in a reduction in the number of precancer cells present in the subject. An effective dose here refers to that amount of the S-antioxidant compound sufficient to result in selective apoptosis of precancer cells. In a preferred embodiment, the S-antioxidant is topically administered. The precancer cells in which apoptosis can selectively be induced include, but are not limited to cells of the type described in Section 5.2.1., below.

The methods of the present invention are also useful for selectively inducing apoptosis of cancer cells by administering a safe and effective amount of an S-antioxidant to a subject. Administration results in a reduction in the number of cancer cells present in the subject. An effective dose here refers to that amount of the S-antioxidant compound sufficient to result in selective apoptosis of cancer cells and, preferably, a regression of precancer or cancer lesions. In a preferred embodiment, the S-antioxidant is topically administered. The cancer cells which in which apoptosis can selectively be induced include, but are not limited to cells of the type and/or disorders described in Section 5.2.2., below.

The methods of the present invention are also useful for reducing the number of cancer cells present in a subject by administering an S-antioxidant to the subject as an adjunct to chemotherapy or radiation therapies such that the susceptibility of the cancer cells to apoptosis is enhanced relative to the non-cancer cells of the subject. S-antioxidant administration can be performed on a subject undergoing or has undergone chemotherapeutic or radiotherapeutic therapies. The time frame between treatment will vary according to the individual. The cancer cells which in which apoptosis can selectively be induced include, but are not limited to cells of the type and/or disorders described in Section 5.2.2., below.

These methods of the present invention are also useful as an adjuncts to p53 therapy, including p53 gene therapy. S-antioxidant administration can be performed on a subject undergoing or has undergone p53 gene therapy. Such an adjunct to p53 therapy can include, first, administration of pharmaceutical S-antioxidant compositions as described in Section 5.1.2., above, which further comprise a functional p53 polypeptide. In one embodiment, the p53 polypeptide comprises a full length, wild-type human p53 polypeptide. In another embodiment, the p53 polypeptide comprises a portion of a human p53 polypeptide which exhibits p53 function.

Methods for using the S-antioxidant compositions of the invention as adjuncts to p53 gene therapy comprise administration of the S-antioxidant compositions of the invention to a subject undergoing or having undergone p53 gene therapy. Methods for p53 gene therapy are well known to those of skill in the art and can include, for example, WO 97/10007; U.S. Pat. No. 5,573,925; and WO 95/11301, which are hereby incorporated by reference in their entirety. Further, for general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, N.Y.; and Kriegler, 1990, Gene Transfer and Expression, A Laboratory Manual, Stockton Press, N.Y.

Still further, the methods of the present invention can be practiced as set forth herein to reduce or inhibit tumor vascularization, to induce differentiation in cancer cells, or to inhibit HIV-1 replication.

In another embodiment of the invention, an S-antioxidant compound of the invention can be administered to treat hyperproliferative or benign dysproliferative disorders. Specific embodiments are directed to treatment or prevention of cirrhosis of the liver (a condition in which scarring has overtaken normal liver regeneration processes), treatment of keloid (hypertrophic scar) formation (disfiguring of the skin in which the scarring process interferes with normal renewal), psoriasis (a common skin condition characterized by excessive proliferation of the skin and delay in proper cell fate determination), benign tumors, fibrocystic conditions, tissue hypertrophy (e.g., prostatic hyperplasia), atherosclerosis, a proliferation of smooth muscle cells lining blood vessels, restenosis, neointimal hyperplasia and mesangial proliferative nephritis.

### 5.2.1. Precancer/Premalignant Conditions

The precancer cells in which apoptosis is induced are generally ones which exhibit at least one functional p53 allele. “Functional” as used herein, refers to an ability of the p53 allele to contribute to differential apoptosis in cells. It is to be noted that in certain instances, administration of the S-antioxidant results in restoration of mutant p53 protein conformation and/or activity to normal. Thus, while precancer cells exhibiting at least one functional p53 allele are preferred targets of the methods of the invention, the methods described herein are not to be limited to such cells.

Precancer cells include, but are not limited to cells which present in conditions known or suspected to precede progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79.)

Hyperplasia is a form of controlled cell proliferation involving an increase in cell number in a tissue or organ, without significant alteration in structure or function. As but one example, endometrial hyperplasia often precedes endometrial cancer.

Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. As but one example, the esophageal metaplasia of Barrett's esophagus often precedes esophageal cancer.

Dysplasia is frequently a forerunner of cancer, and is found mainly in the epithelia; it is the most disorderly form of non-neoplastic cell growth, involving a loss in individual cell uniformity and in the architectural orientation of cells.

Dysplastic cells often have abnormally large, deeply stained nuclei, and exhibit pleomorphism. Dysplasia characteristically occurs where there exists chronic irritation or inflammation, and is often found in the skin, cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of therapeutic administration of the S-antioxidant compounds and compositions of the invention.

As mentioned above, such characteristics of a transformed phenotype include morphology changes, looser substratum attachment, loss of contact inhibition, loss of anchorage dependence, protease release, increased sugar transport, decreased serum requirement, expression of fetal antigens, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are pre-neoplastic lesions indicative of the desirability of therapeutic intervention.

In another embodiment, fibrocystic disease (cystic hyperplasia, mammary dysplasia, particularly adenosis (benign epithelial hyperplasia)) is indicative of the desirability of therapeutic intervention.

In other embodiments, a patient which exhibits one or more of the following predisposing factors for malignancy is treated by administration of an effective amount of the S-antioxidant compositions of the invention: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon cancer), benign monoclonal gammopathy (a possible forerunner of multiple myeloma), and a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W.B. Saunders Co., Philadelphia, pp. 112-113) etc.)

In another specific embodiment, an S-antioxidant of the invention is administered to a human patient treat an actinic keratinosis condition.

In another specific embodiment, the presence of sun-damaged skin, characterized by lost elasticity, distended capillaries, and individual disordered keratinocytes is indicative of the desirability of therapeutic intervention. Such sun-damaged skin represents a precursor of precancerous actinic keratinosis.

### 5.2.2. Malignancies

The cancer cells in which apoptosis is induced are generally ones which exhibit at least one functional p53 allele. “Functional” as used herein, refers to an ability of the p53 allele to contribute apoptosis. It is to be noted that in certain instances, administration of the S-antioxidant results in restoration of mutant p53 protein conformation and/or activity to normal. Thus, while cancer cells exhibiting at least one functional p53 allele are preferred targets of the methods of the invention, the methods described herein are not to be limited to such cells.

Such cancer cells arise as part of malignancies and related disorders which include but are not limited to those listed in Table 1 (for a review of such disorders, see Fishman et al., 1985, Medicine, 2d Ed., J. B. Lippincott Co., Philadelphia):

TABLE 1
MALIGNANCIES AND RELATED DISORDERS

Leukemia

acute leukemia

acute lymphocytic leukemia
acute myelocytic leukemia

myeloblastic
promyelocytic
myelomonocytic
monocytic
erythroleukemia

chronic leukemia

chronic myelocytic (granulocytic) leukemia
chronic lymphocytic leukemia

Polycythemia vera
Lymphoma

Hodgkin's disease
non-Hodgkin's disease

Multiple myeloma
Waldenström's macroglobulinemia
Heavy chain disease
Solid tumors

sarcomas and carcinomas

fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon carcinoma
pancreatic cancer
breast cancer
ovarian cancer
prostate cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatocellular carcinoma
bile duct carcinoma
choriocarcinoma
seminoma
embryonal carcinoma
Wilms' tumor
cervical cancer
uterine cancer
testicular tumor
lung carcinoma
small cell lung carcinoma
bladder carcinoma
glioma
astrocytoma
medulloblastoma
craniopharyngioma
ependymoma
pinealoma
hemangioblastoma
acoustic neuroma
oligodendroglioma
menangioma
melanoma
neuroblastoma
retinoblastoma

In specific embodiments, malignancy or dysproliferative changes (such as metaplasias and dysplasias), or hyperproliferative disorders, are treated in the bladder, breast, colon, lung, melanoma, pancreas, skin (including, for example, basal cell carcinomas and squamous cell carcinomas) or uterus. In other specific embodiments, sarcoma or leukemia is treated or prevented.

### 6. EXAMPLE

### Antioxidant Action via p53-mediated Apoptosis

The Example presented herein demonstrates that sulfur-containing antioxidants such as N-acetylcysteine (NAC) and dimercaptopropanol (DMP) induced apoptosis in several transformed cell lines and primary cultures, but not in normal cells. In contrast, chain-breaking antioxidants such as vitamin E lacked this activity. An increased glutathione level was not required for apoptosis; however, all apoptosis-inducing antioxidants elevated total cellular thiol levels. Antioxidant-induced apoptosis required the p53 tumor suppressor gene. NAC elevated p53 expression post-transcriptionally, by increasing the rate of p53 mRNA translation rather than by altering protein stability. These observations indicate a redox sensor for p53 induction in vivo, with additional transformation-specific information being required for apoptosis.

### 6.1. Materials And Methods

Cell Lines. MEF primary mouse embryo fibroblast cells (passage <5) and E1A/Ha-ras-transformed MEF were as in Lowe (18). The 308 papilloma cell line, which has a mutant Ha-ras allele (Strickland, J. E. et al., 1988, Cancer Res. 48) and wild-type p53 (Liu, M. et al., 1995, Oncogene 10:1955-1960) were also utilized. The (Rostein, J. B. et al., 1988, Mutation Research 202:421-427) 1 mouse embryo fibroblast cell line was as in Levine (Harney, D. M. et al., 1991, Genes Dev. 5:2375-2385). BALB/c 3T3 A31 cells were obtained from the American Type Culture Collection (Bethesda, Md.). 308 papilloma cells were maintained in 0.05 mM Ca2+ EMEM medium with 10% fetal bovine serum, and all other cells were grown in DMEM medium with 10% heat-inactivated fetal bovine serum. Cell culture confluence was maintained below 80%.

Chemicals and Cell Treatments. All chemicals were obtained from Sigma, except for Trolox which was purchased from Aldrich. These compounds were freshly dissolved in medium and adjusted to neutral pH if necessary, or (for vitamin E acetate, Trolox, and BHA) first dissolved in ethanol and added to the medium. Cell viability was determined by trypan blue exclusion as a measure of cell death independent of any growth suppression by p53.

Apoptosis Assays. For fixed cells, apoptosis-associated DNA strand breaks were visualized by fluorescent in situ end-labeling as previously described (Ziegler, A. et al., 1994, Nature 372:773-776). For isolated DNA, DNA fragmentation analysis was performed (Lowe, S. W. et al., 1993, Cell 74:957-967).

For flow cytometry analysis, approximately 10⁶ cells per sample were washed with ice-cold PBS and fixed in 95% ethanol. Cells were then resuspended in 1 mg/ml RNase (Sigma) for 30 min at 37° C. and stained with 0.05 mg/ml propidium iodide (Sigma) for 1 h on ice. Flow cytometric analysis was performed with a FACS Vantage flow cytometer (Becton-Dickinson). Cells were excited at 488 nm and the emission was detected through a 630/22 nm band pass filter. A minimum of 10,000 cells were analyzed for each sample. Cell cycle analysis was performed using the Modfit 5.2 software (Verity Software House). Cells were considered to be in apoptosis if they exhibited sub-G1 DNA fluorescence and a forward angle light scatter (FALS) the same as or slightly lower than that of cells in G1 phase (28). Cellular debris was gated out using the electronic threshold.

Northern and Western Blot Analysis. Northern and Western blot analysis were performed as previously described (Liu, M. et al., 1995, Oncogene 10:1955-1960).

Analysis of p53 Protein Synthesis. Cultures of 308 cells were treated in the absence or presence of 20 mM NAC. At 4.5 h post-treatment, cells were incubated with methionine-free medium containing 2% dialyzed and chelexed fetal bovine serum for 0.5 h. At 5 h post-treatment, biosynthetic labeling was initiated by adding 200 μCi of ³⁵S-methionine per ml of methionine-free medium. The labeling was terminated at 5, 10, or 15 min. Throughout the experiment, 20 mM NAC was included in the group of NAC-treated cells. Cells were then washed twice with 10 ml of ice-cold PBS, scraped, and pelleted by centrifugation at 1500 rpm at 4° C. for 5 min. The supernatant was removed, and the cell pellet was lysed in ice-cold cell lysis buffer (0.5% Triton X-100, 300 mM NaCl, 50 mM Tris-HCl, pH 7.4, 10 μg/ml leupeptin, 0.1 mM phenylmethylsulfonyl fluoride). Aliquots of cell lysate containing equal amounts of protein (30 μg) were subjected to immunoprecipitation analysis with anti-p53 antibody PAb122 (25) and Protein A-agarose (GIBCO-BRL). The immunoprecipitated proteins were resolved on a 10% SDS-PAGE gel. The levels of synthesized p53 protein were then determined by densitometric scanning using a Hewlett-Packard ScanJet 4P Scanner and the NIH image 1.59 analysis software.

Analysis of p53 Protein Half-life. Cultures of 308 cells were treated in the absence or presence of 20 mM NAC. After a 3.5 h incubation, cells were incubated with methionine-free medium containing 2% dialyzed and chelexed fetal bovine serum for 0.5 h. Then cells were labeled by adding 100 μCi of ³⁵S-methionine per ml of methionine-free medium for 1 h. At 5 h post-treatment, cells were washed with phosphate-buffered saline and incubated with a chase medium containing a two-fold excess of unlabeled methionine (45 μg/ml) and cysteine (72 μg/ml) for 0, 20, or 40 min. Throughout the experiment, 20 mM NAC was included in the group of NAC-treated cells. Aliquots of each sample lysate were subjected to immunoprecipitation analysis as in the measurement of p53 protein synthesis rate.

Measurement of GSH and Total Thiols. Cells (4×106) were harvested from each sample. The GSH-400 kit (R & D Systems) was used following the manufacturer's instructions.

### 6.2. Results

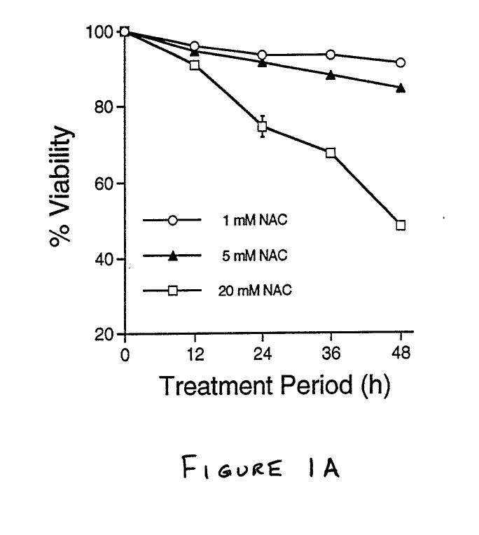

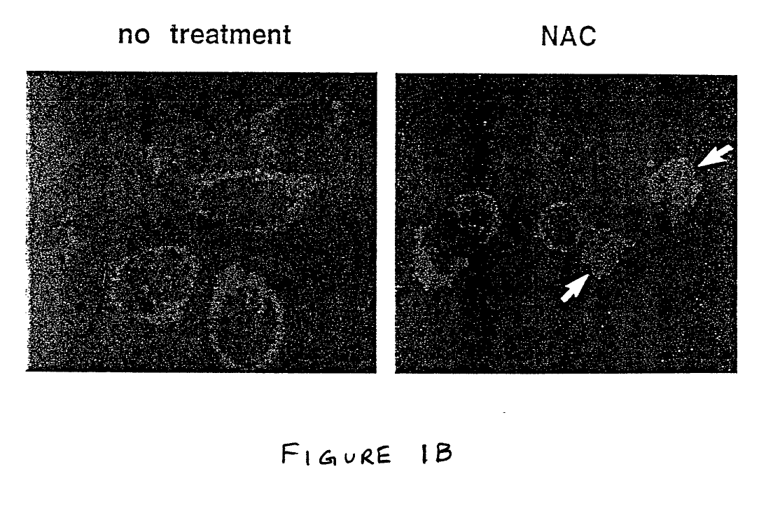

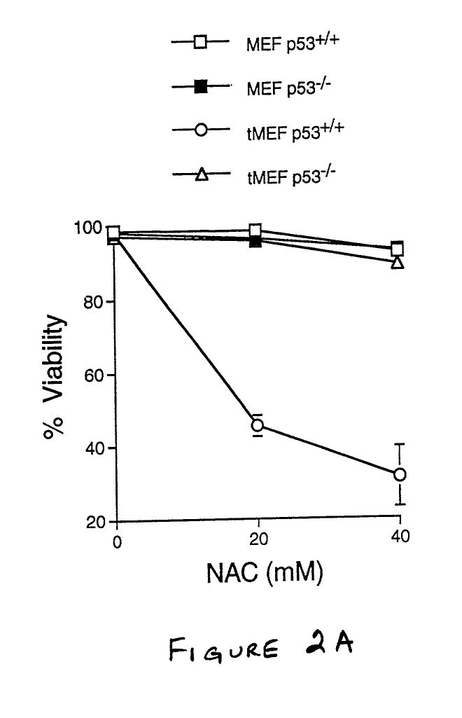

p53-dependent Apoptosis by N-acetylcysteine. Treatment of murine papilloma line 308 cells with the chemopreventive agent NAC led to dose-dependent cell death (FIG. 1A). Death was apoptotic, with cells showing in situ end-labeling of DNA strand-breaks after 24 h treatment with 20 mM NAC, but not at 6 h (data not shown), as well as morphologic changes such as cell shrinkage and nuclear condensation (FIG. 1B). Morphologic changes were minimal in cells treated with doses of NAC associated with high cell viability.

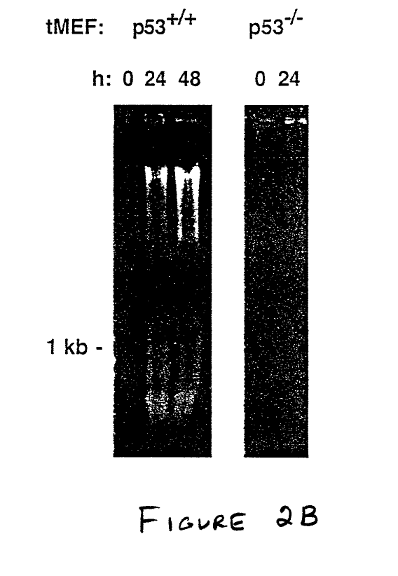

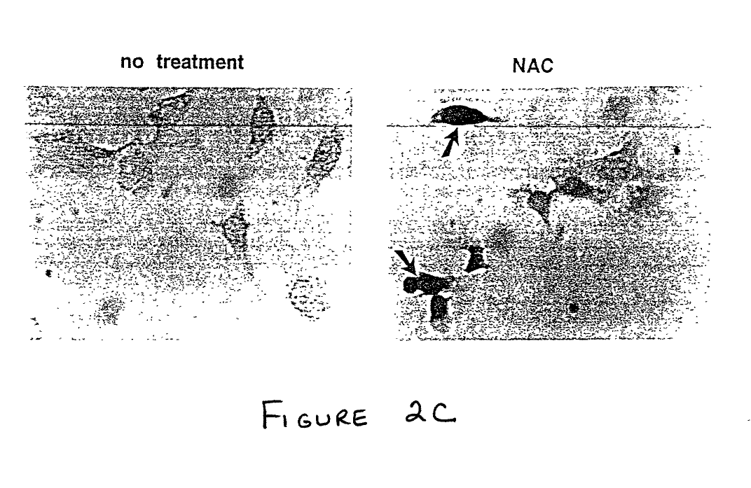

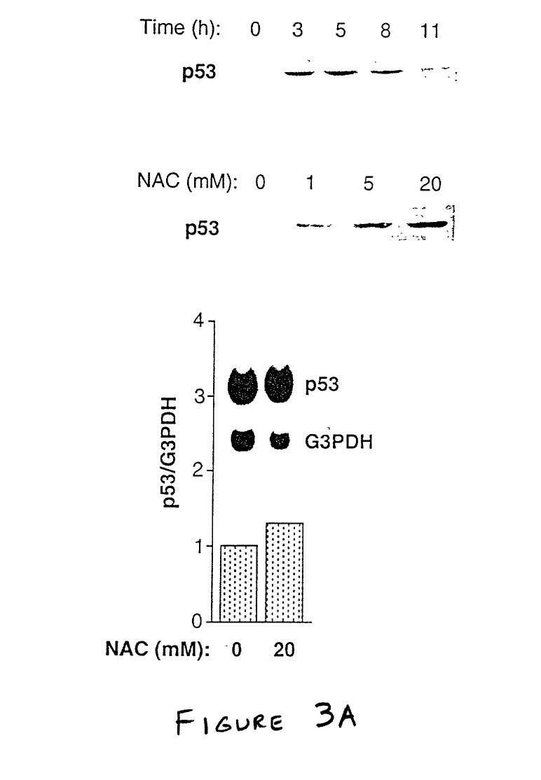

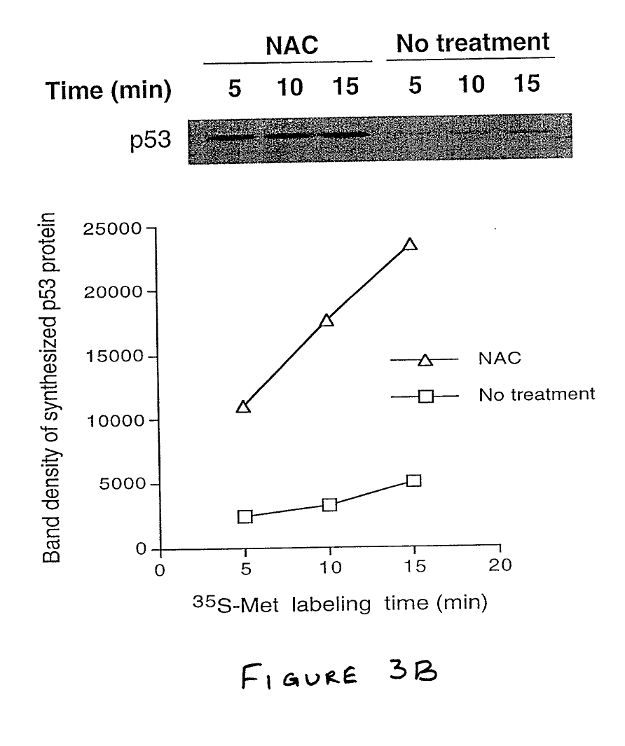

In view of the fact that 308 cells contain a mutant Ha-ras allele and wild-type p53 (Strickland, J. E. et al., 1988, Cancer Res. 48 and Liu, M. et al., 1995, Oncogene 10:1955-19603), a matched pair of normal and transformed cells for comparison was sought. Normal primary MEF cells were compared to a matched line of MEF cells transformed by Ha-ras plus E1A (Lowe, S. W. et al., 1993, Cell 74:957-967). As shown in FIGS. 2A-B, the transformed fibroblasts (tMEF p53^{+/+}) were sensitive to NAC-induced apoptosis, but their normal counterparts (MEF p53^{+/+}) were strikingly resistant. In contrast, both transformed and normal primary cells from p53^{-/-} null mice were deficient in apoptosis induced by NAC (FIG. 2A). A specificity of apoptosis toward transformed cells has been observed previously with chemotherapeutic agents and with hypoxia (Lowe, S. W. et al., 1993, Cell 74:957-967 and Graeber, T. G. et al., 1996, Nature 379:88-91). The specificity of apoptosis toward transformed cells was not due to the level of p53 induction alone, because p53 was induced in their normal counterparts (MEF p53^{+/+}) without causing apoptosis (FIGS. 2A and 2C). An additional, transformation-related, signal is evidently also required for apoptosis. Apoptosis in response to transformation or other heritable abnormalities has been observed in other systems (Lowe, S. W. et al., 1993, Cell 74:957-967; Graeber, T. G. et al., 1996, Nature 379:88-91; Symonds, H. et al., 1994; Cell 78:703-711; Morgenbesser, S. D. et al., 1994, Nature 371:72-74; and Brash, D. E., Nature Medicine 2:525-526).

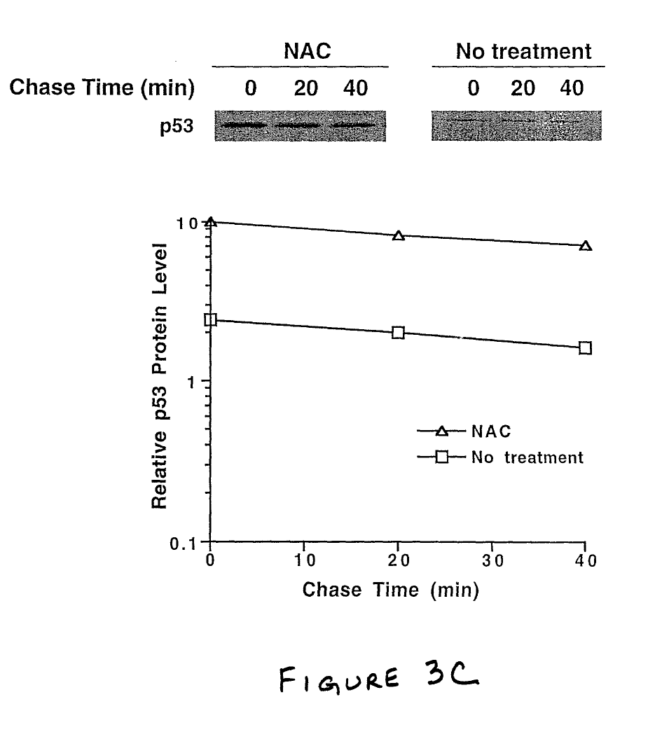

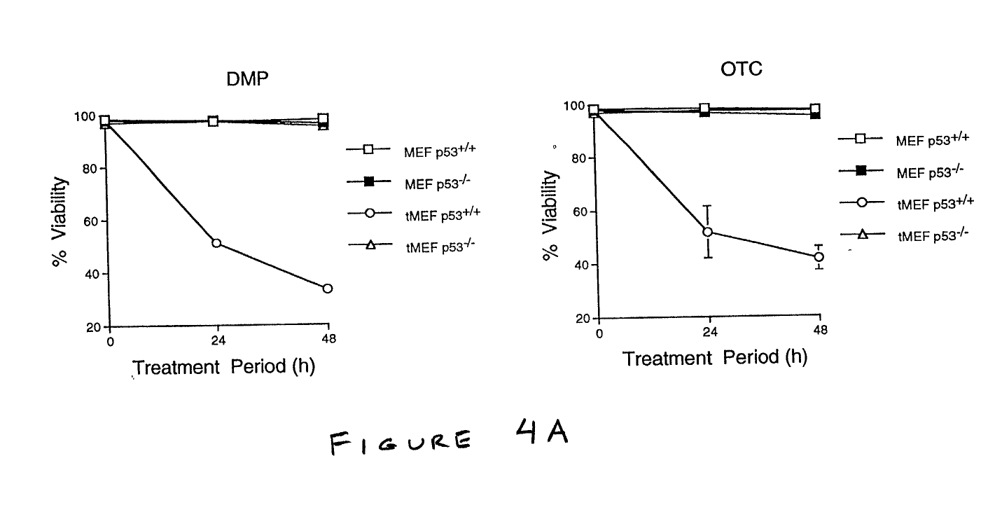

p53 Induction by NAC via Increased p53 Translation Rate. The molecular mediator of antioxidant-induced apoptosis was next investigated. The tumor suppressor protein p53 is required for induction of apoptosis in response to DNA-damaging agents such as g- or UV-irradiation (Lowe, S. W. et al., 1993, Cell 74:957-967 and Ziegler, A. et al., 1994, Nature 372:773-776), and after hypoxia as well (Graeber, T. G. et al., 1996, Nature 379:88-91). As shown in FIG. 3A, treatment of 308 cells with NAC resulted in a dose-dependent 5- to 10-fold increase of p53 protein levels within 3 to 8 hours. Northern blot analysis revealed no major difference in the steady-state level of p53 mRNA between control and NAC-treated cells (FIG. 3A), indicating that p53 induction was controlled at the post-transcriptional level. NAC also induced p53 in the murine fibroblast cell line BALB/c 3T3 A31.

Of all p53-inducing agents, most damage DNA (Levine, A. J., 1997, Cell 88: 323-331). Some of these agents increase p53 post-transcriptionally (Kastan, B. K. et al., 1991, Cancer Res. 51:6304-6311; Fritsche, M. et al., 1993, Oncogene 8:307-318 and Liu, M. et al., 1994, Carcinogenesis 15:1089-1092). In some cases, the induction has been shown to be due to the increased p53 protein stability (Fritsche, M. et al., 1993, Oncogene 8:307-318; Liu, M. et al., 1994, Carcinogenesis 15:1089-1092; Maltzman, W. et al., 1984, Mol. Cell. Biol. 4:1689-1694; and Price, B. D. et al., 1993, Oncogen 8:3055-3062). NAC, in contrast, does not induce DNA damage (Yunis, A. A. et al., 1986, Respiration 50(Suppl):50-55; Chan, J. Y. H. et al., 1986, Carcinogenesis 7:1621-1624 and Solen, G., 1993, Int. J. Radiat. Biol. 64:359-366). In order to determine the precise molecular mechanism(s) for the induction of p53 in response to NAC treatment (FIG. 3A), both the biosynthetic rate of p53 protein and the p53 protein half-life in 308 cells were directly measured. As shown in FIGS. 3B-C, the biosynthetic rate of p53 protein was elevated by nearly 5-fold after NAC treatment. In contrast, the half-life of p53 protein was not altered in the presence of NAC. These results indicate that enhanced translation of p53 mRNA, and not increased protein stability, accounts for the induction of p53 protein following NAC exposure.

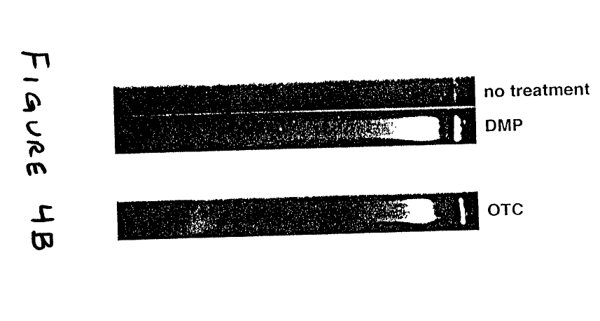

Apoptosis by Other Sulfur-containing Antioxidants. Because NAC is well-known to ameliorate oxidative stress (Flora, S. D. et al., 1992, Cancer Chemoprevention, pp.; Aruoma, O. I. et al., 1989, Free Rad. Biol. Med. 6:593-597, 1989), the capacity of other antioxidants (Anderson, M. E. et al., 1987, Methods in Enzymology 143:313-325; Ceconi, C. et al., 1990, Cardioscience 1:191-198; and Packer, L. et al., 1995, Free Radical Biology & Medicine) for transformation-specific apoptosis was investigated. As demonstrated in FIGS. 4A-C, the sulfur-containing reducing agents 2,3-dimercaptopropanol (DMP) and L-2-oxo-4-thiazolidinecarboxylate (OTC) also selectively induced apoptosis in E1A/Ha-ras transformed cells, but not in their normal counterparts. Lipoic acid behaved similarly. DMP was active at doses as low as 50 μM. DMP, OTC, and lipoic acid also required p53 (FIG. 4A). These agents, as well as NAC, all induced apoptosis in the human p53+/+ colorectal carcinoma cell line RKO.

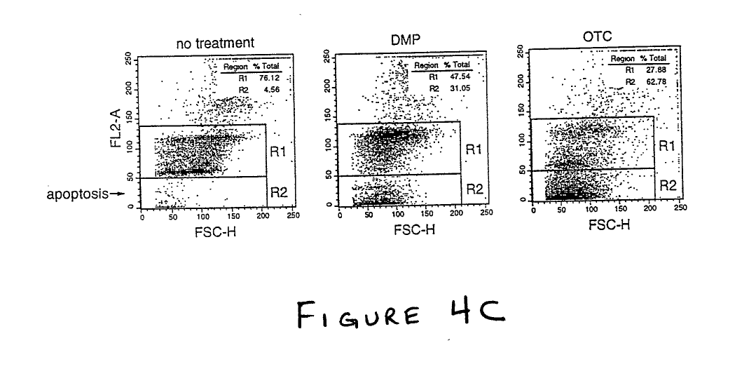

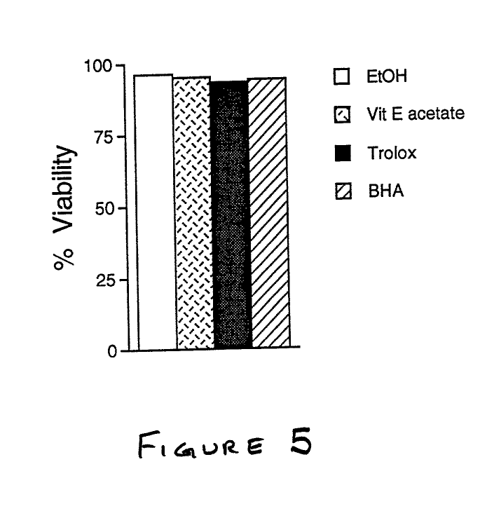

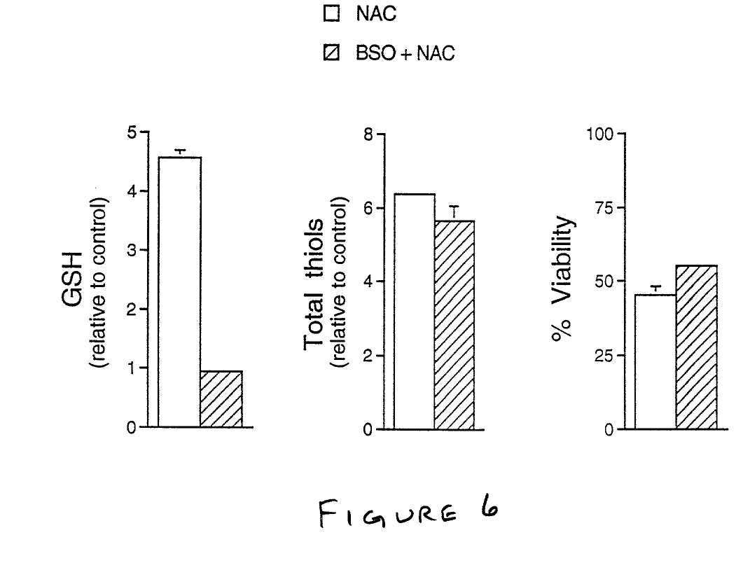

In contrast, the nonsulfur-containing antioxidants vitamin E acetate (tocopherol acetate), BHA, and the water-soluble analog of vitamin E, Trolox (Jacobson, M. D. et al., 1995, Nature 374:814-816), had little effect on cell viability of p53^{+/+} tMEF for at least 48 h (FIG. 5). The chosen antioxidant concentrations here were basically the highest soluble or non-cytotoxic doses to p53^{-/-} tMEF. DNA analysis also confirmed that no apoptosis occurred in p53^{+/+} tMEF cells treated with these chain-breaking antioxidants. Thus, the significant feature of these sulfur-containing compounds appears to be their effect on intracellular redox potential rather than their effect on radical species. In fact, all of the apoptosis-inducing agents tested above elevate cellular thiol levels (FIG. 6).

Glutathione-independence of NAC-induced Apoptosis. A major intracellular pathway of NAC metabolism is deacetylation to the thiol cysteine, the limiting amino acid precursor for synthesis of glutathione (GSH) (Burgunder, J. M. et al., 1989, Eur. J. Clin. Pharmacol.). GSH, in turn, is the major cellular antioxidant (Glutathione: Chemical, Biochemical and Medical Aspects, Vol.). To test the possibility that NAC acts by increasing the level of GSH, we pretreated and co-incubated cells with L-buthionine sulfoximine (BSO); this agent inhibits all GSH synthesis by inactivating g-glutamylcysteine synthetase (Glutathione: Chemical, Biochemical and Medical Aspects, Vol.). As expected, FIG. 6 shows that BSO completely blocks induction of cellular GSH by NAC, while only partially blocking the induction of total thiols. However, BSO did not block NAC-induced apoptosis (FIG. 6), implying that NAC exerts its redox effect directly rather than by increasing GSH.

In this study, it was demonstrated that BSO cannot block NAC-induced apoptosis, although it inhibits cellular GSH elevation by NAC (FIG. 4). This finding indicates that the present apoptosis differs from the BSO-sensitive biphasic toxicity (Fenton reaction) of some antioxidants other than NAC (Held, K. D. et al., 1996, Radiation Research 145:542-553). Furthermore, it was found that penicillamine (50 μM), a potent chelator of copper, had no effect on NAC-induced apoptosis.

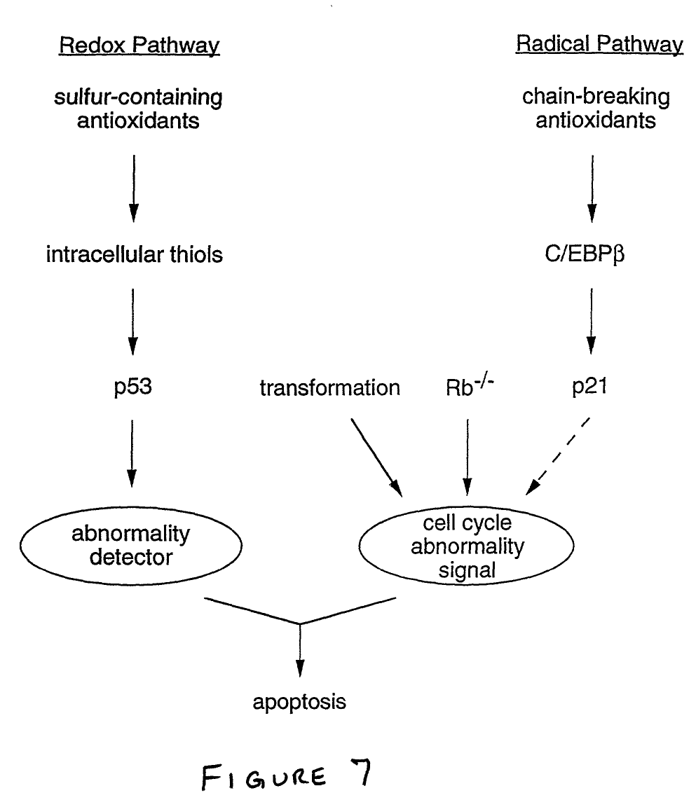

Chain-breaking antioxidants such as vitamin E acetate and Trolox did not induce p53-dependent apoptosis of transformed MEF (FIG. 5). While the p53-dependent mechanism appears to reflect changes in redox potential only, the non-p53 apoptosis pathway appears to involve radical species (Chinery, R. C. et al., 1997, Nature Medicine 3:1233-1241; and Kastan, M. B., 1997, Nature Medicine 3:1192-1193). A possible pathway relating the two mechanisms is shown in FIG. 7.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various references are cited herein, the disclosures of which are incorporated by reference in their entireties.

What is claimed is:

1. A method for selectively inducing apoptosis of precancer cells in a subject, comprising administering to the subject an amount of a sulphur-containing antioxidant effective to selectively induce apoptosis of precancer cells.

2. The method of claim 1, wherein the sulphur-containing antioxidant is administered topically.

3. The method of claim 1, wherein the sulphur-containing antioxidant is administered internally.

4. The method of claim 3, wherein the sulphur-containing antioxidant is administered orally, parenterally or intralesionally.

5. The method of claim 1, wherein the sulphur-containing antioxidant is selected from the group consisting of N-acetylcysteine, 2,3-dimercaptopropanol, L-2-oxo-4-thiazolidinecarboxylate and lipoic acid.

6. The method of claim 1 further comprising administering to the subject a composition comprising a purified p53 polypeptide.

7. The method of claim 1, wherein the subject is undergoing or has undergone p53 gene therapy.

8. The method of claim 1, wherein the precancer cells are actinic keratinocytes.

9. A method of treating a precancer disorder in a subject in need of such treatment, comprising administering to the subject an amount of a sulphur-containing antioxidant effectively to treat the precancer.

10. The method of claim 9, wherein the sulphur-containing antioxidant is administered topically.

11. The method of claim 9, wherein the sulphur-containing antioxidant is administered internally.

12. The method of claim 11, wherein the sulphur-containing antioxidant is administered orally, parenterally or intralesionally.

13. The method of claim 9, wherein the sulphur-containing antioxidant is selected from the group consisting of N-acetylcysteine, 2,3-dimercaptopropanol, L-2-oxo-4-thiazolidinecarboxylate and lipoic acid.

14. The method of claim 9 further comprising administering to the subject a composition comprising a purified p53 polypeptide.

15. The method of claim 9 further comprising administering to the subject an amount of a nucleic acid molecule encoding a p53 polypeptide such that the nucleic acid molecule is expressed in the subject.

16. The method of claim 9, wherein the subject is undergoing or has undergone p53 gene therapy.

17. The method of claim 9, wherein the precancer disorder is actinic keratinosis.

18. A method for selectively inducing apoptosis of cancer cells in a subject, comprising administering to the subject an amount of a sulphur-containing antioxidant effective to selectively induce apoptosis of cancer cells.

19. The method of claim 18, wherein the sulphur-containing antioxidant is administered topically.

20. The method of claim 18, wherein the sulphur-containing antioxidant is administered internally.

21. The method of claim 20, wherein the sulphur-containing antioxidant is administered orally, parenterally or intralesionally.

22. The method of claim 18, wherein the sulphur-containing antioxidant is selected from the group consisting of N-acetylcysteine, 2,3-dimercaptopropanol, L-2-oxo-4-thiazolidinecarboxylate and lipoic acid.

23. The method of claim 18 further comprising administering to the subject a composition comprising a purified p53 polypeptide.

24. The method of claim 18, wherein the cancer cells are melanoma cells, basal cell carcinoma cells, squamous cell carcinoma cells, adenocarcinoma cells, sweat gland carcinoma cells, sebaceous gland carcinoma cells, papillary carcinoma cells or papillary adenocarcinoma cells.

25. The method of claim 18, wherein the subject is undergoing or has undergone a chemotherapeutic treatment for cancer.

26. The method of claim 25, wherein the sulphur-containing antioxidant is administered topically.

27. The method of claim 25, wherein the sulphur-containing antioxidant is administered internally.

28. The method of claim 27, wherein the sulphur-containing antioxidant is administered orally, parenterally or intralesionally.

29. The method of claim 25, wherein the sulphur-containing antioxidant is selected from the group consisting of N-acetylcysteine, 2,3-dimercaptopropanol, L-2-oxo-4-thiazolidinecarboxylate and lipoic acid.

30. The method of claim 25 further comprising administering to the subject a composition comprising a purified p53 polypeptide.

31. The method of claim 25, wherein the cancer cells are melanoma cells, basal cell carcinoma cells, squamous cell carcinoma cells, adenocarcinoma cells, sweat gland carcinoma cells, sebaceous gland carcinoma cells, papillary carcinoma cells or papillary adenocarcinoma cells.

32. The method of claim 18, wherein the subject is undergoing or has undergone a radiotherapeutic treatment.

33. The method of claim 32, wherein the sulphur-containing antioxidant is administered topically.

34. The method of claim 32, wherein the sulphur-containing antioxidant is administered internally.

35. The method of claim 34, wherein the sulphur-containing antioxidant is administered orally, parenterally or intralesionally.

36. The method of claim 32, wherein the sulphur-containing antioxidant is selected from the group consisting of N-acetylcysteine, 2,3-dimercaptopropanol, L-2-oxo-4-thiazolidinecarboxylate and lipoic acid.

37. The method of claim 32 further comprising administering to the subject a composition comprising a purified p53 polypeptide.

38. The method of claim 32, wherein the cancer cells are melanoma cells, basal cell carcinoma cells, squamous cell carcinoma cells, adenocarcinoma cells, sweat gland carcinoma cells, sebaceous gland carcinoma cells, papillary carcinoma cells or papillary adenocarcinoma cells.

39. The method of claim 18, wherein the subject is undergoing or has undergone p53 gene therapy.

40. The method of claim 39, wherein the sulphur-containing antioxidant is administered topically.

41. The method of claim 39, wherein the sulphur-containing antioxidant is administered internally.

42. The method of claim 39, wherein the sulphur-containing antioxidant is administered orally, parenterally or intralesionally.

43. The method of claim 39, wherein the sulphur-containing antioxidant is selected from the group consisting of N-acetylcysteine, 2,3-dimercaptopropanol, L-2-oxo-4-thiazolidinecarboxylate and lipoic acid.

44. The method of claim 39, wherein the cancer cells are melanoma cells, basal cell carcinoma cells, squamous cell carcinoma cells, adenocarcinoma cells, sweat gland carcinoma cells, sebaceous gland carcinoma cells, papillary carcinoma cells or papillary adenocarcinoma cells.

45. A method of treating cancer, comprising administering to a subject in need of such treatment an amount of a sulphur-containing antioxidant effective to treat the cancer.

46. The method of claim 45, wherein the sulphur-containing antioxidant is administered topically.

47. The method of claim 45, wherein the sulphur-containing antioxidant is administered internally.

48. The method of claim 47, wherein the sulphur-containing antioxidant is administered orally, parenterally or intralesionally.

49. The method of claim 45, wherein the sulphur-containing antioxidant is selected from the group consisting of N-acetylcysteine, 2,3-dimercaptopropanol, L-2-oxo-4-thiazolidinecarboxylate and lipoic acid.

50. The method of claim 45 further comprising administering to the subject a composition comprising a purified p53 polypeptide.

51. The method of claim 45 further comprising administering to the subject an amount of a nucleic acid molecule encoding a p53 polypeptide such that the nucleic acid molecule is expressed in the subject.

52. The method of claim 45, wherein the cancer cells are melanoma cells, basal cell carcinoma cells, squamous cell carcinoma cells, adenocarcinoma cells, sweat gland carcinoma cells, sebaceous gland carcinoma cells, papillary carcinoma cells or papillary adenocarcinoma cells.

53. The method of claim 45, wherein the subject is undergoing or has undergone a chemotherapeutic treatment for the cancer.

54. The method of claim 45, wherein the subject is undergoing or has undergone a radiotherapeutic treatment for the cancer.

55. The method of claim 45, wherein the subject is undergoing or has undergone p53 gene therapy.

56. A method for inhibiting HIV replication comprising administering to a subject infected with HIV an amount of a sulphur-containing antioxidant effective to inhibit HIV replication.

57. The method of claim 56, wherein the sulphur-containing antioxidant is selected from the group consisting of N-acetylcysteine, 2,3-dimercaptopropanol, L-2-oxo-4-thiazolidinecarboxylate and lipoic acid.

58. The method of claim 56 further comprising administering to the subject a composition comprising a purified p53 polypeptide.

59. A method for selectively inducing apoptosis of cells of a hyperproliferative or benign dysproliferative disorder in a subject, comprising administering to the subject an effective amount of a sulphur-containing antioxidant.

60. The method of claim 59, wherein the sulphur-containing antioxidant is administered topically.

61. The method of claim 59, wherein the sulphur-containing antioxidant is administered internally.

62. The method of claim 61, wherein the sulphur-containing antioxidant is administered orally, parenterally or intralesionally.

63. The method of claim 59, wherein the sulphur-containing antioxidant is selected from the group consisting of N-acetylcysteine, 2,3-dimercaptopropanol, L-2-oxo-4-thiazolidinecarboxylate and lipoic acid.

64. The method of claim 59 further comprising administering to the subject a composition comprising a purified p53 polypeptide.

65. A method of treating a hyperproliferative or benign dysproliferative disorder, comprising administering to a subject in need of such treatment an amount of a sulphur-containing antioxidant effective to treat the disorder.

66. The method of claim 65, wherein the sulphur-containing antioxidant is administered topically.

67. The method of claim 65, wherein the sulphur-containing antioxidant is administered internally.

68. The method of claim 67, wherein the sulphur-containing antioxidant is administered orally, parenterally or intralesionally.

69. The method of claim 65, wherein the sulphur-containing antioxidant is selected from the group consisting of N-acetylcysteine, 2,3-dimercaptopropanol, L-2-oxo-4-thiazolidinecarboxylate and lipoic acid.

70. The method of claim 65 further comprising administering to the subject a composition comprising a purified p53 polypeptide.

71. A topical formulation comprising a sulphur-containing antioxidant selected from the group consisting of 2,3-dimercaptopropanol, L-2-oxo-4-thiazolidinecarboxylate and lipoic acid, in a cream, ointment, or lotion.

72. The topical formulation of claim 71, wherein the topical formulation further comprises a sunscreen.

73. The topical formulation of claim 71, wherein the topical formulation further comprises a cosmetic.

74. The topical formulation of claim 71, further comprising a p53 polypeptide.

75. The topical formulation of claim 71, further comprising a nucleic acid molecule encoding a p53 polypeptide capable of being expressed in a suitable host cell.

76. A method of preventing a precancer, cancer, hyperproliferative or benign dysproliferative disorder in a human subject, comprising administering to the subject an effective amount of a sulphur-containing antioxidant.

77. The method of claim 76, wherein the sulphur-containing antioxidant is administered topically.

78. The method of claim 76, wherein the sulphur-containing antioxidant is administered internally.

79. The method of claim 78, wherein the sulphur-containing antioxidant is administered orally, parenterally or intralesionally.

80. The method of claim 76, wherein the sulphur-containing antioxidant is selected from the group consisting of N-acetylcysteine, 2,3-dimercaptopropanol, L-2-oxo-4-thiazolidinecarboxylate and lipoic acid.

81. The method of claim 76 further comprising administering to the subject an effective amount of a p53 polypeptide.

82. A pharmaceutical composition comprising (a) an amount of sulphur-containing antioxidant effective to selectively induce apoptosis in a precancer or cancer cell. (b) a purified P53 polypeptide or a purified nucleic acid encoding and capable of expressing a p53 polypeptide in a suitable host cell; and (c) a pharmaceutically acceptable carrier.
